(19) United States
(12) Patent Application Publication
(10) Pub. No.: US 2014/0088517 A1
(43) Pub. Date: Mar. 27, 2014

(54) CATHETERS, ASPIRATORS, AND ATHERECTOMY TOOLS USING VORTICES

(71) Applicant: John Calderone, Powell, OH (US)

(72) Inventor: John Calderone, Powell, OH (US)

(21) Appl. No.: 14/035,724

(22) Filed: Sep. 24, 2013

Related U.S. Application Data
(60) Provisional application No. 61/705,012, filed on Sep. 24, 2012.

(51) Int. Cl. A61M 25/00; A61B 17/3207

(52) U.S. Cl. CPC A61M 25/0023; A61B 17/3207
USPC 604/264; 606/127

(57) ABSTRACT

Catheters, aspirators, and atherectomy tools using vortices are provided.

### CROSS-REFERENCE TO RELATED APPLICATIONS

This application claims priority from U.S. Provisional Patent Application No. 61/705,012, filed on Sep. 24, 2012, which is incorporated by reference herein in its entirety.

### BACKGROUND

In the removal of particulate matter from bodily fluids, it is often difficult to achieve the desired flow of fluid within the collection device (e.g., catheter, aspirator, or atherectomy tool). One common problem is that the collection devices must comprise a flexible tube to channel the particulate and fluid. The integrity of the flexible tube may be compromised if excessive suction forces are applied to the tube, possibly resulting in collapse of the tube and loss of flow altogether.

Inducing vortices in the apertures and/or central lumens of the collection device improves the suction ability of these devices while utilizing the same suction forces as in previous embodiments. Particularly, a vortex flow results in a reduction of a fluid's pressure loss caused by friction between the fluid and the interior of the collection device.

### SUMMARY

In one embodiment, an aspiration catheter is provided, the aspiration catheter comprising: a central lumen; a beveled edge; and a squared edge; wherein at least one of the beveled edge and the squared edge comprise at least one aperture extending into the central lumen; and wherein the at least one aperture is oriented at an angle relative to a longitudinal direction of the catheter.

In another embodiment, an aspiration catheter is provided, the aspiration catheter comprising: a central lumen; at least one of a beveled edge and a squared edge; wherein the at least one of the beveled edge and the squared edge comprise at least one aperture; and wherein the at least one aperture comprises a vortex-inducing spiral feature.

In another embodiment, an aspiration catheter is provided, the aspiration catheter comprising: a central lumen; a beveled edge; and a squared edge; wherein at least one of the beveled edge and the squared edge comprise at least one aperture; and wherein the central lumen comprises a vortex-inducing spiral feature.

In another embodiment, a lung aspirator is provided, the lung aspirator comprising: a central lumen; and a side portion; wherein the side portion comprises an aperture extending into the central lumen; and wherein at least one of the central lumen and the aperture comprises a vortex-inducing spiral feature.

In another embodiment, an atherectomy tool is provided, the atherectomy tool comprising: a central lumen; at least one transversely extending edge; and at least one axially extending edge; wherein at least one of the transversely extending edge and the axially extending edge comprise at least one aperture extending into the central lumen; and wherein the at least one of the central lumen and the at least one aperture comprises a vortex-inducing spiral feature.

In another embodiment, an atherectomy tool is provided, the atherectomy tool comprising: a distal edge; and a proximal edge; wherein the distal edge comprises at least one aperture extending to the proximal edge; and wherein the at least one aperture comprises a vortex-inducing spiral feature.

### BRIEF DESCRIPTION OF THE DRAWINGS

The accompanying figures, which are incorporated in and constitute a part of the specification, illustrate various example apparatuses, and are used merely to illustrate various example embodiments.

FIG. 1 illustrates a cross-section of an example arrangement of an aspiration catheter.

FIG. 2 illustrates an example arrangement of an aspiration catheter.

FIG. 3 illustrates an example arrangement of an aspiration catheter.

FIG. 4 illustrates a cross-section of an example arrangement of a lung aspirator.

FIG. 5 illustrates a cross-section of an example arrangement of an atherectomy tool.

FIG. 6 illustrates a cross-section of an example arrangement of an atherectomy tool.

### DETAILED DESCRIPTION

### Aspiration Catheter

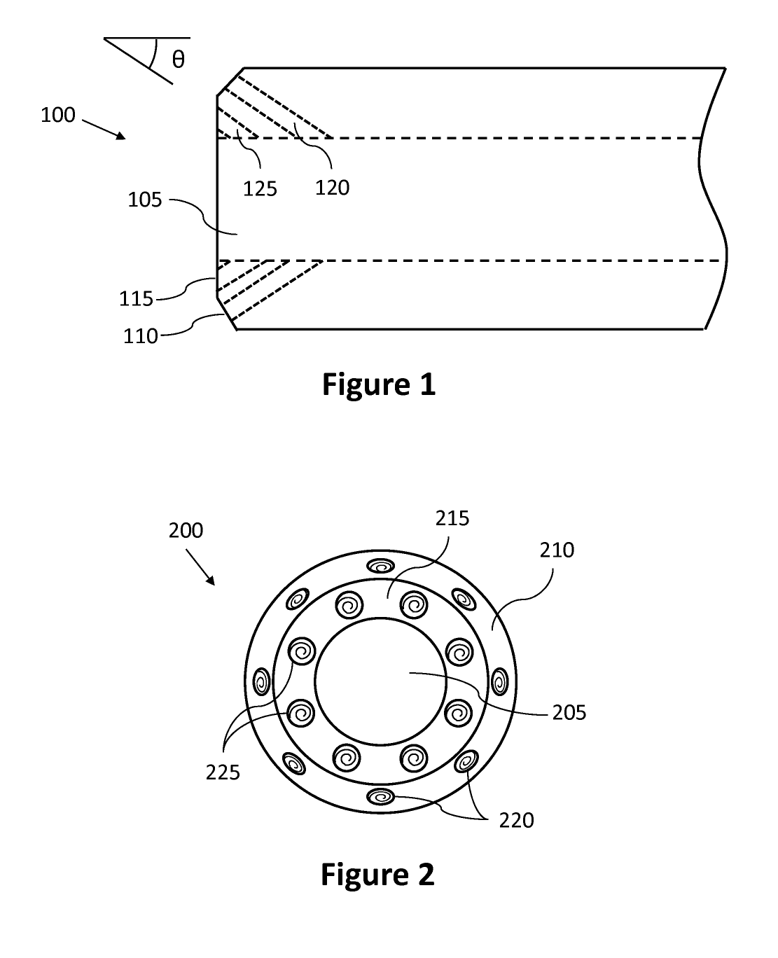

FIG. 1 illustrates a cross-section of an example arrangement of an aspiration catheter 100. Aspiration catheter 100 includes a central lumen 105. Aspiration catheter 100 includes a beveled edge 110 and a squared edge 115. In one embodiment, aspiration catheter 100 may comprise at least one aperture 120 extending from beveled edge 110 into central lumen 105. In another embodiment, aspiration catheter 100 may comprise at least one aperture 125 extending from squared edge 115 into central lumen 105.

In one embodiment, central lumen 105 comprises a hollow channel within aspiration catheter 100. Central lumen 105 may comprise a hollow channel extending longitudinally along the length of aspiration catheter 100. In one embodiment, central lumen 105 is operatively connected to a vacuum pump configured to provide suction through central lumen 105. In another embodiment, central lumen 105 is operative connected at aspiration catheter 100's proximal end to a vacuum source.

Aspiration catheter 100 may comprise a beveled edge 110. Aspiration catheter 100 may be substantially cylindrical in shape, and beveled edge 110 may extend about the periphery of aspiration catheter 100. In one embodiment, beveled edge 110 comprises a chamfered edge. In another embodiment, beveled edge 110 comprises a filleted edge.

Aspiration catheter 100 may comprise a squared edge 115. Squared edge 115 may extend be substantially transversely across aspiration catheter 100. In one embodiment, squared edge 115 comprises a diameter that is less than the total diameter of aspiration catheter 100.

In one embodiment, aspiration catheter 100 comprises at least one aperture 120, 125 extending from its outside surface to central lumen 105.

In one embodiment, beveled edge 110 comprises at least one aperture 120 extending from beveled edge 110 into central lumen 105. In another embodiment, beveled edge 110 comprises a plurality of apertures 120 oriented about beveled edge 110 of aspiration catheter 100. In another embodiment, plurality of apertures 120 are substantially evenly spaced about the circumference of beveled edge 110.

In one embodiment, squared edge 115 comprises at least one aperture 125 extending from squared edge 115 into central lumen 105. In another embodiment, squared edge 115 comprises a plurality of apertures 125 oriented about squared edge 115 of aspiration catheter 100. In another embodiment, plurality of apertures 125 are substantially evenly spaced about the circumference of squared edge 115.

In one embodiment, apertures 120 and 125 are oriented at an angle θ, measured from an axis substantially longitudinal to aspiration catheter 100. Angle θ may be measured from the outside surface of aspiration catheter 100 and extending radially inward of the outside surface of aspiration catheter 100. That is, as illustrated in FIG. 1, apertures 120 and 125 may be angled radially inwardly to central lumen 105. In one embodiment, angle θ is less than about 75 degrees. In another embodiment, angle θ is between about 18.75 degrees and about 56.25 degrees. In another embodiment, angle θ is between about 27.5 degrees and about 47.5 degrees. In another embodiment, angle θ is between about 35 degrees and about 40 degrees.

In one embodiment, at least a portion of central lumen 105 comprises at least one vortex-inducing spiral feature oriented about its interior wall. The vortex-inducing spiral feature may comprise a spiral groove, ridge, or both. That is, central lumen 105 may comprise a grooved and/or ridged interior wall oriented similar female screw threads. In another embodiment, at least a portion of central lumen 105 comprises a plurality of vortex-inducing spiral features. In another embodiment, a distal portion of central lumen 105 comprises at least one vortex-inducing spiral feature oriented about its interior wall. In one embodiment, the at least one vortex-inducing spiral feature is configured in a clockwise orientation. In another embodiment, the at least one vortex-inducing spiral feature is configured in a counterclockwise orientation.

In one embodiment, the at least one vortex-inducing spiral feature is configured to create at least one vortex in a fluid flowing through central lumen 105. In one embodiment, a fluid enters central lumen 105 at the distal end of aspiration catheter 100 and engages the at least one vortex-inducing spiral feature oriented about the interior wall of central lumen 105, thus creating a vortex within the fluid. In one embodiment, the fluid enters central lumen 105 pulled by a vacuum source operatively connected to central lumen 105. In one embodiment, the fluid is a liquid. In another embodiment, the fluid is a bodily fluid, such as blood. In another embodiment, the fluid is a fluid with a particulate matter contained therein. In another embodiment, the fluid is a mixture of solid particles and a liquid or gas. In another embodiment, the fluid is a gas. In one embodiment, the vortex flow of fluid is utilized to decrease the friction of the fluid with the central lumen 105, and thus increase flow of fluid through central lumen 105.

In one embodiment, at least a portion of at least one aperture 120 comprises at least one vortex-inducing spiral feature oriented about its interior wall. That is, at least one aperture 120 may comprise a grooved and/or ridged interior wall oriented similar female screw threads. In another embodiment, at least a portion of at least one aperture 120 comprises a plurality of vortex-inducing spiral features. In another embodiment, a distal portion of at least one aperture 120 comprises at least one vortex-inducing spiral feature oriented about its interior wall. In one embodiment, the at least one vortex-inducing spiral feature is configured in a clockwise orientation. In another embodiment, the at least one vortex-inducing spiral feature is configured in a counterclockwise orientation.

In one embodiment, the at least one vortex-inducing spiral feature is configured to create at least one vortex in a fluid flowing through at least one aperture 120. In one embodiment, a fluid enters at least one aperture 120 at the distal end of aspiration catheter 100 and engages the at least one vortex-inducing spiral feature oriented about the interior wall of at least one aperture 120, thus creating a vortex within the fluid. In one embodiment, the fluid enters at least one aperture 120 pulled by a vacuum source operatively connected to at least one aperture 120 through central lumen 105. In one embodiment, the fluid is a liquid. In another embodiment, the fluid is a bodily fluid, such as blood. In another embodiment, the fluid is a fluid with a particulate matter contained therein. In another embodiment, the fluid is a mixture of solid particles and a liquid or gas. In another embodiment, the fluid is a gas. In one embodiment, the vortex flow of fluid is utilized to decrease the friction of the fluid with the at least one aperture 120, and thus increase flow of fluid through at least one aperture 120.

In one embodiment, at least a portion of at least one aperture 125 comprises at least one vortex-inducing spiral feature oriented about its interior wall. That is, at least one aperture 125 may comprise a grooved and/or ridged interior wall oriented similar female screw threads. In another embodiment, at least a portion of at least one aperture 125 comprises a plurality of vortex-inducing spiral features. In another embodiment, a distal portion of at least one aperture 125 comprises at least one vortex-inducing spiral feature oriented about its interior wall. In one embodiment, the at least one vortex-inducing spiral feature is configured in a clockwise orientation. In another embodiment, the at least one vortex-inducing spiral feature is configured in a counterclockwise orientation.

In one embodiment, the at least one vortex-inducing spiral feature is configured to create at least one vortex in a fluid flowing through at least one aperture 125. In one embodiment, a fluid enters at least one aperture 125 at the distal end of aspiration catheter 100 and engages the at least vortex-inducing spiral feature oriented about the interior wall of at least one aperture 125, thus creating a vortex within the fluid. In one embodiment, the fluid enters at least one aperture 125 pulled by a vacuum source operatively connected to at least one aperture 125 through central lumen 105. In one embodiment, the fluid is a liquid. In another embodiment, the fluid is a bodily fluid, such as blood. In another embodiment, the fluid is a fluid with a particulate matter contained therein. In another embodiment, the fluid is a mixture of solid particles and a liquid or gas. In another embodiment, the fluid is a gas. In one embodiment, the vortex flow of fluid is utilized to decrease the friction of the fluid with the at least one aperture 125, and thus increase flow of fluid through at least one aperture 125.

FIG. 2 illustrates an example arrangement of an aspiration catheter 200, comprising a central lumen 205, a beveled edge 210, and a squared edge 215. Aspiration catheter 200 further comprises at least one aperture 220 extending from beveled edge 210 to central lumen 205. Aspiration catheter 200 additionally comprises at least one aperture 225 extending from squared edge 215 to central lumen 205. As illustrated in FIG. 2, apertures 220 and 225 may comprise a vortex-inducing spiral feature configured to induce a vortex in a fluid flowing through apertures 220 and 225.

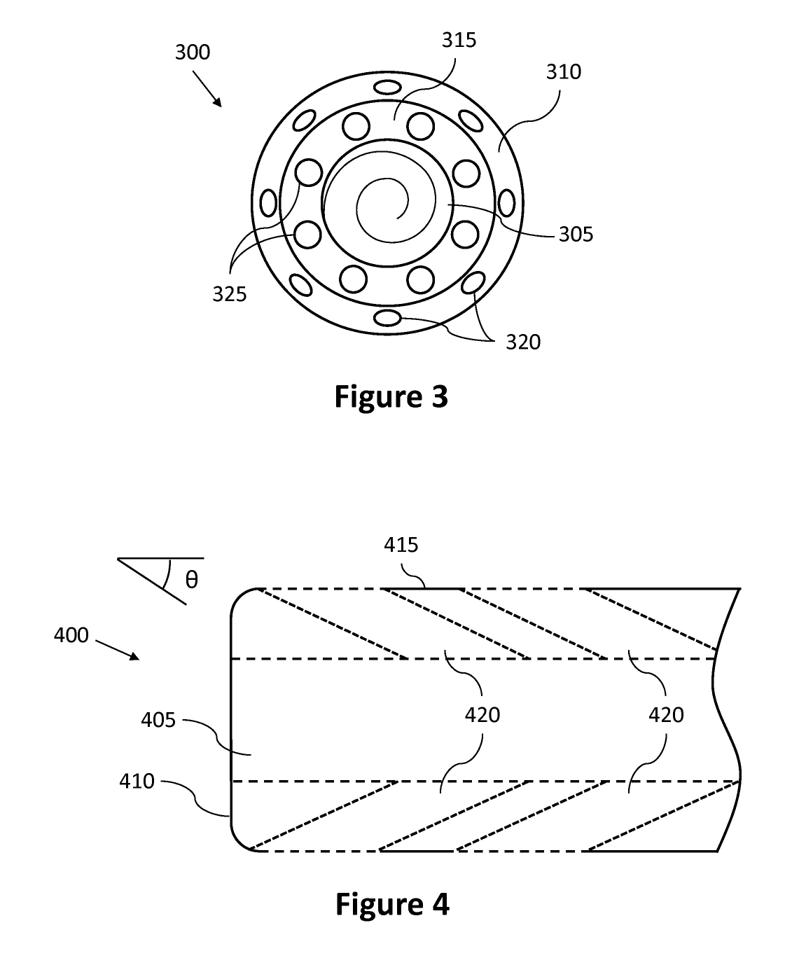

FIG. 3 illustrates an example arrangement of an aspiration catheter 300, comprising a central lumen 305, a beveled edge 310, and a squared edge 315. Aspiration catheter 300 further comprises at least one aperture 320 extending from beveled edge 310 to central lumen 305. Aspiration catheter 300 additionally comprises at least one aperture 325 extending from squared edge 315 to central lumen 305. As illustrated in FIG. 3, central lumen 305 may comprise a vortex-inducing spiral feature configured to induce a vortex in a fluid flowing through central lumen 305.

In one embodiment, any combination of central lumen 305 and apertures 320 and 325 may comprise at least vortex-inducing spiral feature configured to induce a vortex in a fluid flowing through central lumen 305 and apertures 320 and 325.

In one embodiment, any of the aspiration catheters described herein can be utilized as intravascular devices that aspirate liberated debris from percutaneous interventions at various anatomic sites. In another embodiment, any of the aspiration catheters described herein can be utilized in angioplasty procedures. In another embodiment, the central lumen of any of the aspiration catheters described herein may additionally provide a passage for guidewires, balloons, nets, or other interventional components.

### Lung Aspirator

FIG. 4 illustrates a cross-section of an example arrangement of a lung aspirator 400. Lung aspirator 400 comprises a central lumen 405, a front portion 410, and a side portion 415. Side portion 415 may comprise at least one aperture 420 extending from side portion 415 into central lumen 405. In one embodiment, at least a portion of central lumen 405 comprises at least one vortex-inducing spiral feature. In another embodiment, at least one of the at least one aperture 420 comprises at least one vortex-inducing spiral feature.

In one embodiment, at least one aperture 420 is oriented at an angle θ, measured from and axis substantially longitudinal to lung aspirator 400. Angle θ may be measured from the outside surface of lung aspirator 400 and extending radially inward of the outside surface of lung aspirator 400. That is, as illustrated in FIG. 4, aperture 420 may be angled radially inwardly to central lumen 405. In one embodiment, angle θ is less than about 75 degrees. In another embodiment, angle θ is between about 18.75 degrees and about 56.25 degrees. In another embodiment, angle θ is between about 27.5 degrees and about 47.5 degrees. In another embodiment, angle θ is between about 35 degrees and about 40 degrees.

In one embodiment, any combination of central lumen 405 and at least one aperture 420 may comprise at least vortex-inducing spiral feature configured to induce a vortex in a fluid flowing through central lumen 405 and at least one aperture 420.

In one embodiment, lung aspirator 400 can be used to remove particulate and/or fluid from lung tissue via suction. Suction from lung aspirator 400 may be provided by a vacuum source operatively connected to lung aspirator 400. In one embodiment, central lumen 405 may additionally provide a passage for guidewires, balloons, nets, or other interventional components.

### Atherectomy Tool

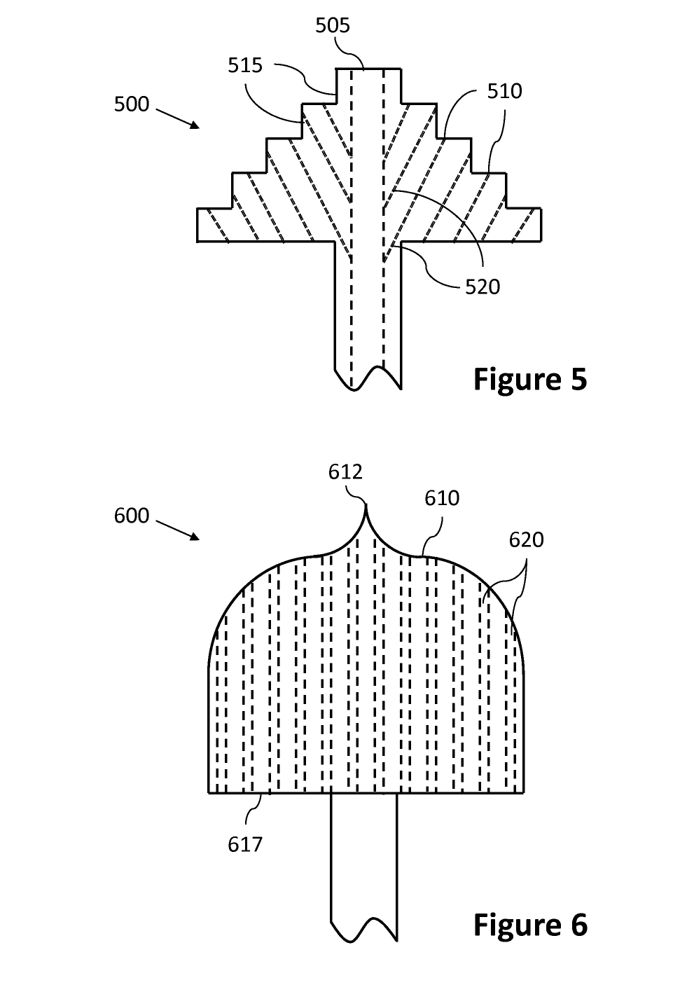

FIG. 5 illustrates a cross-section of an example arrangement of an atherectomy tool 500. Atherectomy tool 500 comprises a central lumen 505, at least one transversely extending edge 510, and at least one axially extending edge 515. At least one transversely extending edge 510 and at least one axially extending edge 515 comprise at least one aperture 520 extending from at least one transversely extending edge 510 and at least one axially extending edge 515 into central lumen 505. In one embodiment, at least a portion of central lumen 505 comprises at least one vortex-inducing spiral feature. In another embodiment, at least one of the at least one aperture 520 comprises at least one vortex-inducing spiral feature. In one embodiment, at least one transversely extending edge 510 and/or at least one axially extending edge 515 comprise diamonds oriented on the surface of at least one transversely extending edge 510 and/or at least one axially extending edge 515 to facilitate greater cutting ability.

In one embodiment, any combination of central lumen 505 and at least one aperture 520 may comprise at least vortex-inducing spiral feature configured to induce a vortex in a fluid flowing through central lumen 505 and at least one aperture 520.

FIG. 6 illustrates a cross-section of an example arrangement of an atherectomy tool 600. Atherectomy tool 600 comprises a distal edge 610, a distal point 612, and a proximal edge 617. Distal edge 610 comprises at least one aperture 620 extending from distal edge 610 to proximal edge 617. In one embodiment, at least one of at least one aperture 620 comprises a vortex-inducing spiral feature. In another embodiment, atherectomy tool 600 comprises a central lumen (not shown). The central lumen may comprise a vortex-inducing spiral feature.

In one embodiment, any combination of the central lumen (not shown) and at least one aperture 620 may comprise at least vortex-inducing spiral feature configured to induce a vortex in a fluid flowing through the central lumen (not shown) and at least one aperture 620. In one embodiment, at least one of the central lumen (not shown) and at least one aperture 620 is operatively connected to a vacuum source.

In one embodiment, atherectomy tool 500, 600 can be utilized for rheolytic (mechanical) removal of arterial plaque. In another embodiment, atherectomy tool 500, 600 can be used to enhance of efficacy of percutaneous transluminal angioplasty (PTA) procedures by improving removal of arterial plaque. Difficult and/or calcified atherosclerotic plaques can cause vessel occlusion that may not be able to be rectified via angioplasty and stenting. For such situations, atherectomy devices can be utilized. Atherectomy tool 500, 600 can be rotated similar to a drill bit and contacted to the plaque, causing the plaque to break up into particulate. The particulate can be drawn through one or both of central lumen 505 (not illustrated in FIG. 6) and at least one aperture 520, 620, utilizing a vacuum source operatively connected to one or both of central lumen 505 (not illustrated in FIG. 6) and at least one aperture 520, 620.

To the extent that the term “includes” or “including” is used in the specification or the claims, it is intended to be inclusive in a manner similar to the term “comprising” as that term is interpreted when employed as a transitional word in a claim. Furthermore, to the extent that the term “or” is employed (e.g., A or B) it is intended to mean “A or B or both.” When the applicants intend to indicate “only A or B but not both” then the term “only A or B but not both” will be employed. Thus, use of the term “or” herein is the inclusive, and not the exclusive use. See Bryan A. Garner, A Dictionary of Modern Legal Usage 624 (2d. Ed. 1995). Also, to the extent that the terms “in” or “into” are used in the specification or the claims, it is intended to additionally mean “on” or “onto.” To the extent that the term “substantially” is used in the specification or the claims, it is intended to indicate a nature of an element and/or a relationship between elements within a reasonable degree of precision and tolerance as is acceptable in the relevant field of technology. To the extent that the term “selectively” is used in the specification or the claims, it is intended to refer to a condition of a component wherein a user of the apparatus may activate or deactivate the feature or function of the component as is necessary or desired in use of the apparatus. To the extent that the term “operatively connected” is used in the specification or the claims, it is intended to mean that the identified components are connected in a way to perform a designated function. To the extent that the term “horizontal” or “vertical” is used in the specification or the claims, it is intended to mean that the identified components are substantially horizontal or substantially vertical, respectively, when installed in a human that is standing in an upright position. As used in the specification and the claims, the singular forms “a,” “an,” and “the” include the plural. Finally, where the term “about” is used in conjunction with a number, it is intended to include ±10% of the number. In other words, “about 10” may mean from 9 to 11.

As stated above, while the present application has been illustrated by the description of embodiments thereof, and while the embodiments have been described in considerable detail, it is not the intention of the applicants to restrict or in any way limit the scope of the appended claims to such detail. Additional advantages and modifications will readily appear to those skilled in the art, having the benefit of the present application. Therefore, the application, in its broader aspects, is not limited to the specific details, illustrative examples shown, or any apparatus referred to. Departures may be made from such details, examples, and apparatuses without departing from the spirit or scope of the general inventive concept.

1. An aspiration catheter comprising:
a central lumen;
a beveled edge; and
a squared edge;
wherein at least one of the beveled edge and the squared edge comprise at least one aperture extending into the central lumen; and
wherein the at least one aperture is oriented at an angle relative to a longitudinal direction of the catheter.

2. The aspiration catheter of claim 1, wherein the at least one aperture is oriented at an angle of less than about 75 degrees.

3. The aspiration catheter of claim 1, wherein the at least one aperture is oriented at an angle between about 18.75 degrees and about 56.25 degrees.

4. The aspiration catheter of claim 1, wherein the at least one aperture is oriented at an angle between about 27.5 degrees and about 47.5 degrees.

5. The aspiration catheter of claim 1, wherein the at least one aperture comprises a vortex-inducing spiral feature.

6. The aspiration catheter of claim 1, wherein the central lumen comprises a vortex-inducing spiral feature.

7. An aspiration catheter comprising:
a central lumen; and
at least one of a beveled edge and a squared edge;
wherein the at least one of the beveled edge and the squared edge comprise at least one aperture; and
wherein the at least one aperture comprises a vortex-inducing spiral feature.

8. The aspiration catheter of claim 7, wherein the central lumen comprises a vortex-inducing spiral feature.

9. The aspiration catheter of claim 7, wherein the at least one aperture is oriented at an angle relative to a longitudinal direction of the catheter.

10. The aspiration catheter of claim 9, wherein the at least one aperture is oriented at an angle of less than about 75 degrees.

11. The aspiration catheter of claim 9, wherein the at least one aperture is oriented at an angle between about 18.75 degrees and about 56.25 degrees.

12. The aspiration catheter of claim 9, wherein the at least one aperture is oriented at an angle between about 27.5 degrees and about 47.5 degrees.

13. A lung aspirator comprising:
a central lumen; and
a side portion;
wherein the side portion comprises an aperture extending into the central lumen; and
wherein at least one of the central lumen and the aperture comprises a vortex-inducing spiral feature.

14. The lung aspirator of claim 13, wherein the aperture is oriented at an angle relative to a longitudinal direction of the lung aspirator.

15. The lung aspirator of claim 14, wherein the aperture is oriented at an angle of less than about 75 degrees.

16. The lung aspirator of claim 14, wherein the aperture is oriented at an angle between about 18.75 degrees and about 56.25 degrees.

17. The lung aspirator of claim 14, wherein the aperture is oriented at an angle between about 27.5 degrees and about 47.5 degrees.

18. An atherectomy tool comprising:
a central lumen;
at least one transversely extending edge; and
at least one axially extending edge;
wherein at least one of the transversely extending edge and the axially extending edge comprise at least one aperture extending into the central lumen; and
wherein the at least one of the central lumen and the at least one aperture comprises a vortex-inducing spiral feature.

19. The atherectomy tool of claim 18, wherein at least one of the transversely extending edge and the axially extending edge comprise diamonds oriented on a surface of at least one of the transversely extending edge and the axially extending edge.

20. The atherectomy tool of claim 18, wherein the atherectomy tool is configured to be rotated and contacted to an arterial plaque.
